(19) United States
(12) Patent Application Publication
(10) Pub. No.: US 2016/0151527 A1
(43) Pub. Date: Jun. 2, 2016

(54) Ionic Liquids

(71) Applicant: The Procter & Gamble Company, Cincinnati, OH (US)

(72) Inventors: Lynette Anne Makins HOLLAND, Abbots Langley (GB); Oreste TODINI, Bruxelles (BE); David Michael EIKE, West Chester, OH (US); Jose Maria VELAZQUEZ MENDOZA, Ascot (GB); Sarah Anne TOZER, London (GB); Pauline Mary MCNAMEE, Camberley (GB); Henry Charles Reginald FOVARGUE, London (GB); Judith Ann GREGORY, Fleet (GB); Kenneth Richard SEDDON, Belfast (GB); Harambage Quintus Nimal GUNARATNE, Belfast (GB); Alberto Vaca PUGA, Belfast (GB); Julien ESTAGER, Belfast (GB); Feng-Ling WU, Belfast (GB); Shane Declan DEVINE, Belfast (GB)

(21) Appl. No.: 15/010,034

(22) Filed: Jan. 29, 2016

Related U.S. Application Data
(63) Continuation of application No. PCT/US15/52086, filed on Sep. 25, 2015.
(60) Provisional application No. 62/054,991, filed on Sep. 25, 2014.

(51) Int. Cl. A61L 9/01; C11D 3/34; C07D 291/06; C07C 217/08; C07C 309/04; C07C 211/08; C11D 3/30; C11D 3/00

(52) U.S. Cl. CPC A61L 9/01; C11D 3/30; C11D 3/3409; C11D 3/349; C11D 3/3472; C11D 3/0015; C07C 217/08; C07C 309/04; C07C 211/08; C07D 291/06; C11D 3/3481

(57) ABSTRACT

The present invention relates to a novel ionic liquid composition comprising a novel cation and process for making and using thereof.

### FIELD OF THE INVENTION

The present invention relates to compositions comprising an ionic liquid comprising a novel cation and process for making and using said compositions.

### BACKGROUND OF THE INVENTION

Ionic liquids (“ILs”) are salts that are liquid at temperatures below 100° C., preferably at ambient or near ambient temperatures (i.e., from 15° C. to 35° C.). Due to the lower melting points, ionic liquids can act as solvents for reactions and because ionic liquids are made of ions rather than neutral molecules, such reactions provide distinct selectivities and reactivities as compared to conventional organic solvents. Also, unlike conventional organic solvents, ionic liquids essentially do not have a vapour pressure. This means that they do not evaporate and therefore emit no undesirable vapors into the atmosphere. Ionic liquids are able to dissolve a wide range of organic compounds, and this property can be fine tuned according to the cation or anion of the ionic liquids.

Typically, ionic liquids have found applications in electrochemistry uses, such as for example, fuel cells, solar cells, electrodeposition processes and other electrochemical applications, ionic liquids have also been shown to be effective in chemical separation and extraction (US2004/133058, Arlt W. et al.). More recently, ionic liquids have found applications in consumer product formulations and industrial product formulations for surface treating, air treating, cleaning and other benefits (US2004/077519, Price K. et al.).

For the aforementioned reasons, there remains a need to discover new ionic liquids with similar and/or additional advantages. It is desirable if such ionic liquids can be made by simple processes with low amounts of waste and impurities. It is also desirable that such ionic liquids be suitable for consumer applications (e.g., for the house or the automobile) and/or industrial applications.

### SUMMARY OF THE INVENTION

Advantageously, new ionic liquids have been discovered. In one aspect, the present invention provides ionic liquids containing a novel cation, and various ionic liquids based products/compositions thereof. In another aspect of the present invention, processes for manufacturing the ionic liquids and/or compositions comprising the ionic liquids are provided. In yet another aspect of the present invention, a method for treating a target surface or air with compositions comprising the ionic liquids is provided. These and other features of the present invention will become apparent to one skilled in the art upon review of the following detailed description when taken in conjunction with the appended claims.

### DETAILED DESCRIPTION OF THE INVENTION

### Definitions

As used herein, articles such as “a” and “an” when used in a claim, are understood to mean one or more of what is claimed or described.

As used herein, the terms “include”, “includes” and “including” are meant to be non-limiting.

As used herein, the term “consumer products” or “consumer applications” refers to a material that is used by a user (i.e., a consumer) in, on or around their person, house (e.g., kitchen surfaces, bathroom surfaces, carpets, floors, windows, mirrors and countertops), car (e.g., automobile interiors, automobile exterior, metal surfaces and windshields), and other personal or household articles (e.g., dishware, fabrics, cookware, utensils, tableware and glassware). “Consumer products” may also include the material used by institutional users (e.g., hotels, restaurants, offices) or by service providers (e.g., commercial dry cleaners and janitorial services).

As used herein, the term “industrial products” or “industrial applications” refers to a material that is used in a commercial process of making an article. Non-limiting examples include degreasing compositions for degreasing articles, such as metals, plastics and wood products; and textile treating compositions for processing and/or finishing textiles into fabric articles, such as garments, draperies and the like.

As used herein, the term “treating” refers to a composition or a process of cleaning, refreshing or maintaining the target surface or air. For example, “treating” includes the processes of imparting a pleasant odour to a fabric article, air, skin or a hard surface, or removing the wrinkled or worn appearance from a fabric article.

As used herein, the term “surface”, “targeted surface” or “treated surface” refers to an inanimate, non-biological surface. Non-limiting examples of such surfaces are found in soft surfaces such as fabrics, fabric articles, textiles, fibers; and hard surfaces such as dishware, cookware, utensils, glassware, countertops, kitchen, surfaces, bathroom surfaces, floors, windows, car interior and exterior, metals and combinations thereof. These terms also include biological surfaces such as hair, skin or teeth.

Certain chemical groups named here are preceded by a shorthand notation indicating the total number of carbon atoms that are to be found in the indicated chemical group. For example: C₁-C₂₀alkyl describes an alkyl group having a total of 1 to 20 carbon atoms. The total number of carbons in the shorthand notation does not include carbons that may exist in substituents of the group described. Unless specified to the contrary, the following terms have the following meaning:

“Amino” refers to the —NH₂ functional group.

“Azido” refers to the —N₃ functional group.

“Cyano” refers to the —CN functional group.

“Halo” refers bromo, chloro, fluoro or iodo.

“Halide” refers to a halo atom bearing a negative charge such as for example, bromide (Br⁻), chloride (Cl⁻), fluoride (F⁻) or Iodide (I⁻).

“Hydroxyl” refers to —OH functional group.

“Nitro” refers to the —NO₂ functional group.

“Oxo” refers to the ═O substituent.

“Alkyl” refers to a group containing straight or branched hydrocarbon chain consisting solely of carbon and hydrogen atoms, containing no unsaturation, having from 1 to 20 carbon atoms, preferably 1 to 12 carbon atoms, preferably 1 to 8 or preferably 1 to 6 carbon atoms, e.g., methyl, ethyl, n-propyl, 1-methylethyl(iso-propyl), n-butyl, n-pentyl and the like. An alkyl group may be optionally substituted.

“Alkenyl” refers to a group containing straight or branched hydrocarbon chain consisting solely of carbon and hydrogen atoms, containing at least one carbon-carbon double bond, having from 2 to 20 carbon atoms, preferably 2 to 12 carbon atoms, or preferably 1 to 8 carbon atoms, e.g., ethenyl, prop-2-enyl, but-1-enyl, pent-1-enyl, penta-1,4-dienyl and the like. An alkenyl group may be optionally substituted.

“Alkynyl” refers to a group containing straight or branched hydrocarbon chain consisting solely of carbon and hydrogen atoms, containing at least one carbon-carbon triple bond, having from 2 to 20 carbon atoms, preferably 2 to 12 carbon atoms, or preferably 1 to 8 carbon atoms, e.g., ethynyl, propynyl, butynyl, pentynyl, hexynyl and the like. An alkynyl group may be optionally substituted.

“Alkylene” or “ alkylene chain” refers to a group containing straight or branched hydrocarbon chain linking the rest of the molecule to a group, consisting solely of carbon and hydrogen, containing no unsaturation and having from 1 to 12 carbon atoms, e.g., methylene, ethylene, propylene, n-butylene and the like. An alkylene chain may be optionally substituted.

“Alkenylene” or “alkenylene chain” refers to a group containing straight or branched hydrocarbon chain linking the rest of the molecule to a group, consisting solely of carbon and hydrogen, containing at least one carbon-carbon double bond and having from 2 to 20 carbon atoms, preferably 2 to 12 carbon atoms, e.g., ethenylene, propenylene, n-butenylene and the like. An alkenylene chain may be optionally substituted.

“Alkynylene” or “alkynylene chain” refers to a group containing straight or branched hydrocarbon chain linking the rest of the molecule to a group, consisting solely of carbon and hydrogen, containing at least one carbon-carbon triple bond and having from 2 to 20 carbon atoms, e.g., propynylene, n-butynylene, and the like. An alkynylene chain may be optionally substituted.

“Alkoxy” refers to a functional group of the formula —ORₐ where Rₐ is an alkyl chain as defined above containing 1 to 20 carbon atoms, preferably 1 to 12 carbon atoms.

“Alkoxyalkyl” refers to a functional group of the formula —Rₐ₁—O—Rₐ₂ where Rₐ₁ is an alkylene chain as defined above and Rₐ₂ is an alkyl chain as defined above containing 1 to 20 carbon atoms, preferably 1 to 12 carbon atoms.

“Oligo-alkoxyalkyl” refers to a functional group containing more than 2 oxygen atoms that are separated by a straight or branched alkyl chain as defined above containing at least 2 or more carbon atoms, e.g., etherated alkyl chains.

“Aryl” refers to aromatic monocyclic or multicyclic hydrocarbon ring system consisting only of hydrogen and carbon and containing from 6 to 18 carbon atoms, preferably 6 to 10 carbon atoms, where the ring system may be partially saturated. Non-limiting example of aryls include: fluorenyl, phenyl and naphthyl. The term “aryl” or the prefix “ar” (such as in “aralkyl”) is meant to include aryl that may be optionally substituted.

“Arylene” refers to a linking aryl group, and where the aryl as defined above.

“Cycloalkyl” refers to a stable non-aromatic mono-cyclic or polycyclic hydrocarbon group consisting solely of carbon and hydrogen atoms, which may include fused or bridged ring systems, having from 3 to 15 carbon atoms, preferably having from 3 to 10 carbon atoms or preferably from 3 to 7 carbon atoms, and which is saturated or unstaturated. A cycloalkyl may be optionally substituted.

“Cycloalkylalkyl” refers to a functional group of the formula —RₐR_{d} where Rₐ is an alkylene as defined above and R_{d} is a cycloalkyl as defined above.

“Haloalkyl” refers to an alkyl as defined above that is substituted by one or more halo groups, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl and the like.

“Heterocyclyl” refers to a stable 3- to 18-membered non-aromatic ring which consists of 2 to 20 carbon atoms and from 1 to 6 heteroatoms selected from the group consisting of nitrogen, oxygen and sulfur. The heterocyclyl may be a monocyclic, bicyclic, tricyclic or tetracyclic ring system, which may include fused or bridged ring systems; and the nitrogen, carbon or sulfur atoms in the heterocyclyl may be optionally oxidized; the nitrogen atom may be optionally quaternized; and the heterocyclyl may be partially or fully saturated. A heterocyclyl may be optionally substituted.

“Heterocyclylalkyl” refers to a functional group of the formula —RₐRₑ where Rₐ is an alkylene as defined above and Rₑ is a heterocyclyl as defined above, and if the heterocyclyl is a nitrogen-containing heterocyclyl, the heterocyclyl may be attached to the alkylene at the nitrogen atom.

“Heteroaryl” refers to a 5- to 18-membered aromatic ring which consists of 1 to 17 carbon atoms and from 1 to 10 heteroatoms selected from the group consisting of nitrogen, oxygen and sulfur. The heteroaryl may be a monocyclic, bicyclic, tricyclic or tetracyclic ring system, which may include fused or bridged ring systems; and the nitrogen, carbon or sulfur atoms in the heteroaryl may be optionally oxidized; the nitrogen atom may be optionally quaternized. A heteroaryl may be optionally substituted.

“Heteroarylalkyl” refers to a functional group of the formula —RₐR_{f} where Rₐ is an alkylene as defined above and R_{f} is a heteroaryl as defined above.

“Heteroarylene” refers to a linking heteroaryl group and where the heteroaryl is as defined above.

“Optionally substituted” means that the subsequently described event of circumstances may or may not occur and that the description includes instances where the event or circumstance occurs and instances in which it does not. For example, unless specified otherwise, “optionally substituted aryl” means that the chemical moiety may or may not be substituted by one or more of the following groups: alkyl, alkenyl, halo, haloalkenyl, cyano, nitro, aryl, cycloalkyl, heterocyclyl, heteroaryl, oxo, —OR¹⁰, —OC(O)—R¹⁰, —NR(¹⁰)², —C(O)R¹⁰, —C(O)OR¹⁰, —C(O)N(R¹⁰)₂, —N(R¹⁰)C(O)OR¹², —N(R¹⁰)C(O)R¹², —N(R¹⁰)S(O)ₜR¹² (where t is 1 to 2), —S(O)ₜOR¹² (where t is 1 to 2), —S(O)ₓR¹² (where x is 0 to 2) and —S(O)ₜN(R¹⁰)₂ (where t is 1 to 2) where each R¹⁰ is independently hydrogen, alkyl, haloalkyl, cycloalkyl, cycloalkylalkyl, aryl (optionally substituted with one or more halo groups), aralkyl, heterocyclyl, heterocyclylalkyl, heteroaryl or heteroarylalkyl; and each R¹² is alkyl, haloalkyl, cycloalkyl, cycloalkylalkyl, aryl, aralkyl, heterocyclyl, heterocyclylalkyl, heteroaryl or heteroarylalkyl, and where each of the above substituents is unsubstituted unless otherwise indicated.

In all embodiments of the present invention, all percentages are by weight of the total fragrance composition, as evident by the context, unless specifically stated otherwise. All ratios are weight ratios, unless specifically stated otherwise, and all measurements are made at 25° C., unless otherwise designated.

### Ionic Liquids

As used herein, the term “ionic liquids” refer to liquids which consist exclusively of ions and is present in a liquid form at temperatures lower than 100° C., preferably at ambient or room temperature (i.e., from 15° C. to 35° C.). Ionic liquids of the present invention may be used for consumer and/or industrial applications. Particularly preferred ionic liquids are suitable for use in consumer products and have to be chosen so as to exclude an adverse effect in terms of health or ecology on people, nature and the environment. For example, consumer products, such as for example, household cleaners or air fresheners, which may come into direct contact with humans must be safe.

Ionic liquids have no effective vapour pressure (i.e., essentially zero) and may be easy to handle. Their polarity can be readiably adjusted so as to be suitable to a wide range of applications. Furthermore, ionic liquids are odourless and will not impart an odour of their own when added into the products of the present invention. Particularly preferable ionic liquids are ones where the solutes are fully miscible to form a single phase liquid.

Typically, ionic liquids can have high viscosities (i.e., greater than about 1,000 mPa·s) at room temperature. The high viscosities can be problematic in formulating the consumer products of the present invention. Therefore, in an embodiment, the present invention is preferably directed to ionic liquids (undiluted with adjuncts, co-solvents or free water) which have viscosities of less than about 1000 mPa·s, preferably less than 750 mPa·s, preferably less than about 500 mPa·s, most preferably less than 200 mPa·s as measured at 20° C. In some embodiments, the viscosity of the undiluted ionic liquids are in the range from about 0.1 mPa·s to about 400 mPa·s, preferably from 0.2 mPa·s to about 300 mPa·s, and more preferably from about 0.5 mPa·s to about 250 mPa·s.

The viscosities of the ionic liquids and compositions containing therein can be measured on a Brookfield viscometer model number LVDVII+ at 20° C., with Spindle S31 at the appropriate speed to measure materials of differing viscosities. Typically, the measurement is performed at speed from 12 rpm to 60 rpm. The undiluted state is prepared by storing the ionic liquids in a desiccator containing a desiccant (e.g. anyhydrous calcium chloride) at room temperature for at least about 48 hours prior to the viscosity measurement. This equilibration period unifies the amount of innate water in the undiluted samples.

In one aspect, the ionic liquid composition of the present invention has at least one salt comprising: (a) an anion; and (b) a cation selected from the group consisting of the representative formulae:

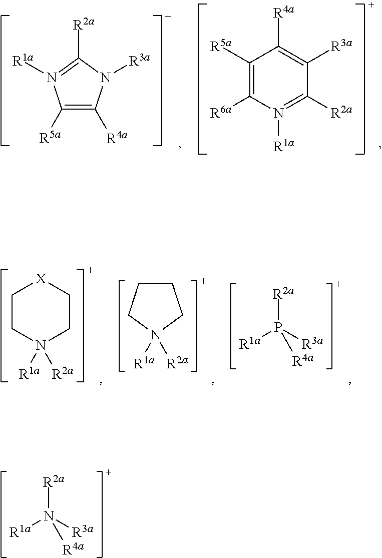

and mixtures thereof;

wherein: X is CH₂ or O;
each R^{1a}, R^{3a}, and R^{4a} are independently selected from hydrogen, C₁-C₂₀alkyl, C₂-C₂₀alkenyl, C₂-C₂₀alkynyl, C₁-C₂₀alkoxy, C₁-C₂₀alkoxyC₁-C₂₀alkyl, C₃-C₇cycloalkyl C₃-C₇cycloalkylC₁-C₄alkyl, C₂-C₂₀heterocyclyl, C₆-C₁₀aryl, C₆-C₁₀arylC₁-C₁₀alkyl, C₁-C₁₀heteroaryl, halo, haloC₁-C₂₀alkyl, hydroxyl, hydroxyC₁-C₂₀alkyl, or —N(R^{6a})₂;
each R^{2a} is independently selected from hydrogen, C₁-C₂₀alkyl, C₁-C₂₀alkenyl, or C₁-C₂₀alkynyl;
each R^{5a} is independently selected from hydrogen, C₁-C₂₀alkyl, C₁-C₂₀alkenyl, C₁-C₂₀alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, or alkylene chain, alkenylene chain, or alkynylene chain; and
each R^{8a} is independently selected from a hydrogen, alkyl, alkenyl or alkynyl.

In one embodiment of the present invention, the cation as set forth above wherein:
R^{1a} and R^{3a} are independently selected from C₁-C₆alkyl;
R^{2a} is C₁-C₆alkyl;
R^{4a} is C₁-C₂₀alkoxyC₁-C₂₀alkyl substituted with —OR¹⁰; and
R¹⁰ is C₁-C₆alkyl;

Of this embodiment of the present invention, wherein the cation is (N-ethyl-2-(2-methoxyethoxy)-N,N-dimethylethanaminium); 2-(2-ethoxyethoxy)-N-ethyl-N,N-dimethylethanaminium; or mixtures thereof.

In another embodiment of the present invention, the cation as set forth above wherein:
R^{2a} and R^{4a} are hydrogen; and
R^{3a} and R^{5a} are C₁-C₆ alkyl.

Of this embodiment of the present invention, wherein the cation is N-benzyl-N,N-dimethyloctan-1-aminium; N-benzyl-N,N-dimethylnonan-1-aminium; or mixtures thereof.

The preparation of the cations are shown below in the Examples.

In yet another embodiment of the present invention, wherein the anion has the formula (I):

[R¹—O—C(O)—CH(SO₃)—R³—C(O)—O—R²]⁻ (I)

wherein:
R¹ and R² are independently selected from the group consisting of alkyl, mono or oligo-alkoxyalkyl or alkenyl; and
R³ is alkylene, heteroarylene, arylene, or cycloalkylene.

Of this embodiment of the present invention, the anion according to formula (I) wherein:
R¹ and R² are independently selected from alkyl; and
R³ is —(CH₂)ₙ— wherein n is an integer of from 1 to 12.

Of this embodiment of the present invention, wherein the anion according to formula (I) is 1,4-bis(2-ethylhexoxy)-1,4-dioxobutane-2-sulfonate (i.e., AOT, docusate or dioctylsulfosuccinate).

In yet another embodiment of the present invention, wherein the anion has the formula (II):

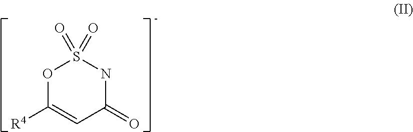

wherein:
R⁴ is selected from the group consisting of hydrogen, cyano, alkyl, alkoxy and alkoxyalkyl.

Of this embodiment of the present invention, wherein the anion according to formula (II) is 6-methyl-3,4-dihydro-1,2,3-oxathiazin-4-one 2,2-dioxide (i.e., acesulfame).

In yet another embodiment of the present invention, wherein the anion has the formula (III):

[R⁹—SO₂—N—SO₂—R⁹]⁻ (III)

wherein:
R⁹ is selected from the group consisting of haloalkyl, alkyl and alkenyl.

Of this embodiment of the present invention, wherein the anion according to formula (III) is bis{(trifluoromethyl)sulfonyl}amide (i.e., [NTf₂]⁻).

In yet another embodiment of the present invention, wherein the anion is selected from the group consisting of the compounds of formula (I), (II), (III), and mixtures thereof.

In yet another embodiment of the present invention, wherein the anion is independently selected from the group consisting of:

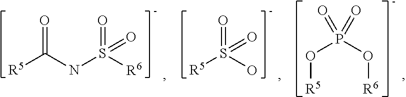

and combinations thereof;

wherein:
each R⁵ and R⁶ are independently selected from the group consisting of hydrogen, alkyl, alkenyl, alkynyl, alkoxyalkyl, cycloalkyl, cycloalkylalkyl, aryl, aralkyl, heterocyclyl, heterocyclyalkyl, heteroaryl and heteroarylalkyl.

In yet another embodiment of the present invention, the ionic liquids, preferably wherein the anion component, may be selectively made to be hydrophobic.

In yet another embodiment of the present invention, the ionic liquids (i.e., cations and anions) are essentially free of any of the following chemical elements: antimony, barium, beryllium, bromine, cobalt, chromium, fluorine, iodine, lead, nickel, selenium, or thallium. By “essentially free” it is meant that no cations or anions containing any of the foregoing chemical elements are intentionally added to form the ionic liquids of the present invention.

The preparation of the anions is generally known and can take place, for example, as described in (Peter Wasserscheid and T. Welton (Eds.), Ionic Liquids in Synthesis, 2^{nd} Edition, Wiley-VCH, 2008). In addition, the anions are also available commercially. Thus, for example, sodium docusate and potassium acesulfame is available from Sigma Aldrich and lithium bistriflamide (LiNTf₂) is available from TCI.

The ionic liquids of the present invention have one or more of the above identified salts. It is understood that the ionic liquids can comprise either a single anionic species and a single cationic species or a plurality of different anionic and cationic species. By using different anionic species and/or different cation species, the properties of the ionic liquids can be matched in an optimal way to the solutes and/or other components of the composition comprising the ionic liquids. In an embodiment of the invention, the ionic liquids consist of more than one anionic species.

### Ionic Liquids Applications

The present invention relates to compositions, consumer products and industrial products comprising the ionic liquids and the methods of using the same in the following applications: personal care cleaning, dish/food cleaning, home care (e.g., kitchen/bath), biofilm removal, dry-cleaning (home and commercial), laundry (pretreatment, cleaning and fabric care), textile processing and finishing, car care (interior and exterior), industrial degreasing and air care. Accordingly, ionic liquids can be present in various compositions suitable for use in various consumer and/or industrial applications including but not limited to a laundry detergent, a dish cleaning detergent, a hard surface cleaning composition, a dry cleaning composition, an air care composition, a beauty care composition, a fragrance composition, a car care composition, a textile treating composition or an industrial degreasing composition. Without wishing to be bound by theory, it is believed that the fundamental chemical and/or physical properties of ionic liquids can be used advantageously in the surface or air treating compositions. In one aspect, ionic liquids have a high solubilizing ability, due to their high polarity and charge density; thus, ILs can be an effective solvent for soils. Therefore, composition containing ionic liquids exhibit enhanced soil removal ability, compared to similar compositions without the ionic liquids.

In another aspect, the functional groups of the ionic liquids can be varied such that the resulting ionic liquids are tuned to the characteristics of the target surface. For example, the functional groups can be selected such that the resulting ionic liquids have the desired hydrophilicity or hydrophobicity to interact more strongly with certain target surfaces. Without wishing to be bound by theory, the mechanism by which ionic liquids can effectively interact with target surfaces include, but not limited to, charge transfer, ion exchange, van der Waals forces, hydrogen bonding and electrostatic interaction.

In still another aspect, the ionic liquids are non-volatile and non-flammable and have high thermal stability. As such, the ionic liquids are particularly suitable for use in surface or air treating compositions for both safety and aesthetic reasons. Due to the fact that ionic liquids have essentially no vapor pressure, compositions containing ionic liquids as the active ingredients or the solvents may reduce or eliminate the problems associated with chemical vapors and will not leave behind unsightly streaks on surfaces treated by them.

The ionic liquids may be present in any desired effective amount, depending on the nature of the intended application. Typically, the ionic liquids are present in an amount ranging from about 0.1 wt % to 99.9 wt %, preferably from about 1 wt % to about 85 wt %, and more preferably from about 5 wt % to about 75 wt % by weight of the composition. In some embodiments, the ionic liquids comprise at least about 50 wt % of the composition.

The ionic liquids containing compositions may be formulated in the form of liquid, gel, paste, foam or absorbed onto a solid or encapsulated into a particle. When the composition is in the solid form, it can be further processed into granules, powders, tablets or bars. The ionic liquids containing compositions may also comprise adjunct ingredients commonly used in air or surface treating compositions used for consumer and/or industrial applications. When present, an adjunct ingredient may comprise from about 0.01 wt % to about 20 wt %, preferably from about 0.1 wt % to about 10 wt % of the composition.

Suitable adjunct ingredients may be selected from the group consisting of enzymes, bleaches, surfactants, perfumes, co-solvents, cleaning agents, anti-bacterial agents, anti-static agents, brighteners, dye fixatives, dye abrasion inhibitors, anti-crocking agents, wrinkle reduction agents, wrinkle resistance agents, soil release polymers, sunscreen agents, anti-fade agents, builders, sudsing agents, composition malodor control agents, dyes, colorants, speckles, pH buffers, waterproofing agents, soil repellency agents and mixtures thereof.

Examples of suitable adjunct ingredients are disclosed in U.S. Pat. No. 6,488,943, Beerse et al.; U.S. Pat. No. 6,514,932, Hubesch et al.; U.S. Pat. No. 6,548,470, Buzzaccarini et al.; U.S. Pat. No. 6,482,793, Gordon et al.; U.S. Pat. No. 5,545,350, Baker et al.; U.S. Pat. No. 6,083,899, Baker et al.; U.S. Pat. No. 6,156,722, Panandiker et al.; U.S. Pat. No. 6,573,234, Sivik et al.; U.S. Pat. No. 6,525,012, Price et al.; U.S. Pat. No. 6,551,986, Littig et al.; U.S. Pat. No. 6,566,323, Littig et al.; U.S. Pat. No. 6,090,767, Jackson et al.; and/or U.S. Pat. No. 6,420,326, Maile et al.

In some embodiments, such as personal cleansing, laundry or dish-washing, ionic liquids containing compositions may be applied to the skin, fabric or dish directly, or may be diluted with water to form a wash liquor, which contacts the skin, fabric or dish. In other embodiments, the ionic liquids containing compositions may be in the form of a liquid, which can be applied to the target surface as a liquid spray, as a unit dose formulation, as an aerosol spray or as a pour-on liquid, which can be poured onto the target surface directly or indirectly via a substrate such as a fibrous web substrate (made by woven, non-woven or knitted technologies), a pulp-based substrate (made by air-felt or wet-laid technologies, including paper towels and tissues), a sponge, or a foam substrate. Another mode of use would be to incorporate ionic liquids containing compositions into or onto these substrates (e.g., impregnated in a wipe or a mitten).

### EXAMPLES

The following examples are provided to further illustrate the present invention and are not to be construed as limitations of the present invention, as many variations of the present invention are possible without departing from its spirit or scope.

The structures of the ionic liquids of the present invention are characterized by techniques well-known to the skilled person, including for example: ¹H NMR (nuclear magnetic resonance) spectroscopy, ¹³C NMR spectroscopy, halogen analysis and CHN elemental analysis.

Nuclear magnetic resonance (“NMR”) spectroscopy is a spectroscopic technique well-known to the skilled person and used herein to characterise the ionic liquids prepared herein.

Mass Spectrometry (“MS”) is a spectrometric technique used herein to quantify the mass to charge ratio of particles or molecules. Two different methods of MS were used electron spray MS (“ES-MS”) and electron ionisation MS (“EI-MS”). ES-MS is used for non-volatile materials such as the ionic liquids. EI-MS is used for volatile materials such as the precursor materials.

### Example 1

### Syntheses of Ionic Liquids

The general method for synthesising ionic liquids of the present invention consists of: (i) synthesis of a chloride or sulfonate ester precursor; (ii) quaternisation of an amine using a chloroalkane or sulfonyl ester in order to obtain an ionic liquid with chloride or sulfonate anions; and (iii) metathesis (i.e., anion exchange) reaction in order to create the target ionic liquid. This is illustrated in Reaction Scheme 1.

### Reaction Scheme 1

### General Synthesis of Targeted Ionic Liquids

(i) Precursor synthesis step: R—OH+SOCl₂→R—Cl
   R—OH+R′SO₂Cl→R′SO₃R
(ii) Quaternisation step: R—Cl+Amine→[Cation]Cl
   R′SO₃R+Amine→[Cation][R′SO₃]
(iii) Metathesis Step: [Cation]Cl+M[Anion]→[Cation][Anion]+MCl
   where M=Na or K

Ionic liquids are formed by combining salts of cations and anions (e.g., the sodium or potassium salt of the anion and a chloride salt of the cation). Different ionic liquids can be synthesised such that the interactions between the ionic liquids and the solutes (i.e., PRMs) are optimised, preferably to provide for a positive deviation from Raoult's Law (Atkins, P. W. and Paula, J. D., Atkins' Physical Chemistry, 9^{th} Edit. (Oxford University Press Oxford, 2010). Ionic liquids lend themselves to preparation via combinatorial or high throughput chemistry. The steps shown in the Reaction Scheme 1 are described below in more details.

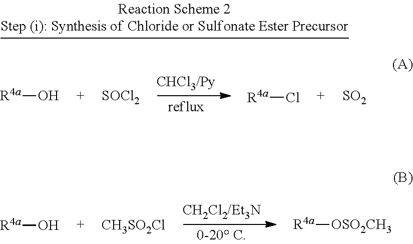

(A) Chloride Precursor Synthesis:

Equimolar amounts of 2-(2-methoxyethoxy)ethanol (1A) or 2-(2-ethoxyethoxy)ethanol (1B) and pyridine are added to a three-neck round bottom flask under N₂. Trichloromethane is used as a solvent for the reaction. Thionyl chloride (1.2 mol eq) is added dropwise to the stirred mixture via a pressure equalising funnel. Once the addition is completed, the reaction mixture is then heated at 60° C. under reflux for 24 h. The reaction mixture is then washed with H₂O (4×), saturated aqueous NaHCO₃ (3×), dried over anhydrous MgSO₄ and purified by filtration. The solvent is removed under reduced pressure and the resulting crude product is then distilled yielding the pure product.

(B) Sulfonate Ester Precursor Synthesis:

Equimolar amounts of 2-(2-methoxyethoxy)ethanol (1A) and triethylamine in dichloromethane are added to a round-bottom flask in an ice bath under N₂. The mixture is stirred at 0° C. for 20 min before sulfonyl chloride is added dropwise, in slight excess, via a pressure equalising funnel. Once the addition is completed, the reaction mixture is warmed to room temperature overnight. The reaction mixture is then washed with H₂O (6×), saturated aqueous NaCl solution (3×), dried over anhydrous MgSO₄, purified by filtration and concentrated to yield the sulfonate ester precursor. Sulfonate ester precursor is obtained as a colourless liquid by fractional distillation of the crude product.

TABLE 1
Structures of 1A and 1B

| | Chemical Name | Chemical Structure |
|---|---|---|
| 1A | 2-(2-methoxyethoxy)ethanol | 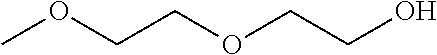 |
| 1B | 2-(2-ethoxyethoxy)ethanol | 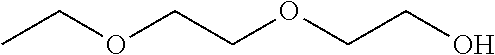 |

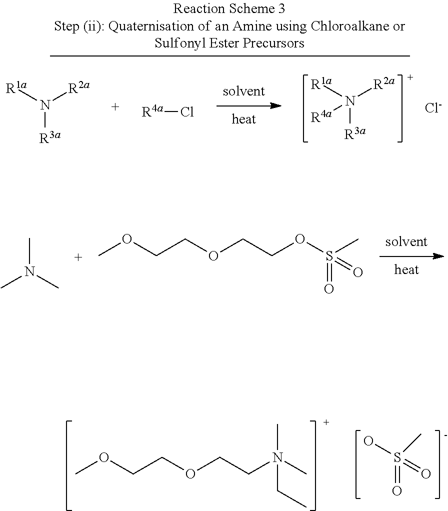

Equimolar amounts of chloride precursor or sulfonyl ester precursor and amine (dimethylethylamine or dimethyloctylamine) are added to a tetrahydrofuran in a sealable reactor. The sealed reaction mixture is stirred and heated at 60° C. until the reaction is completed. The progress of reaction is monitored by NMR spectroscopy. Solvent and unreacted amine are removed under reduced pressure. The product is washed with ethyl ethanoate (6×) and cyclohexane (2×). The residual solvent is removed via rotary evaporator and the product is dried under high vacuum at 40-80° C. for 1-3 days. Exemplary ionic liquids in Table 2 are synthesised according to this method.

TABLE 2
Exemplary Ionic Liquids with Chloride Anion or Methylsulfonate Anion from
Quaternisation Reaction

| Example | Chemical Name | Chemical Structure |
|---|---|---|
| Ionic Liquid 1 | (N-ethyl-2-(2- methoxyethoxy)-N,N- dimethylethanaminium) chloride | 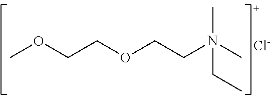 |
| Ionic Liquid 2 | 2-(2-ethoxyethoxy)-N-ethyl- N,N-dimethylethanaminium chloride | 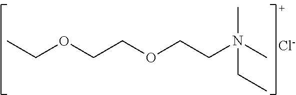 |
| Ionic Liquid 3 | (N-ethyl-2-(2- methoxyethoxy)-N,N- dimethylethanaminium) methyl sulfonate | 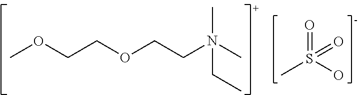 |
| Ionic Liquid 4 | N-benzyl-N,N- dimethylnonan-1-aminium chloride | 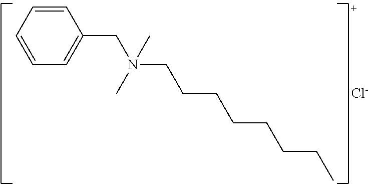 |
| Ionic Liquid 5 | 2-(2-ethoxyethoxy)-N-ethyl- N,N-dimethylethanaminium methylsulfonate | 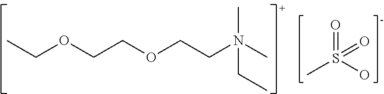 |
| Ionic Liquid 6 | N-benzyl-N,N- dimethylnonan-1-aminium methylsulfonate | 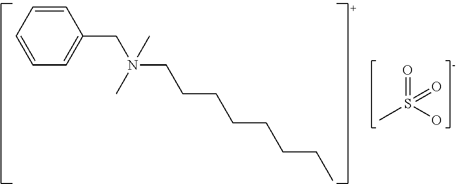 |

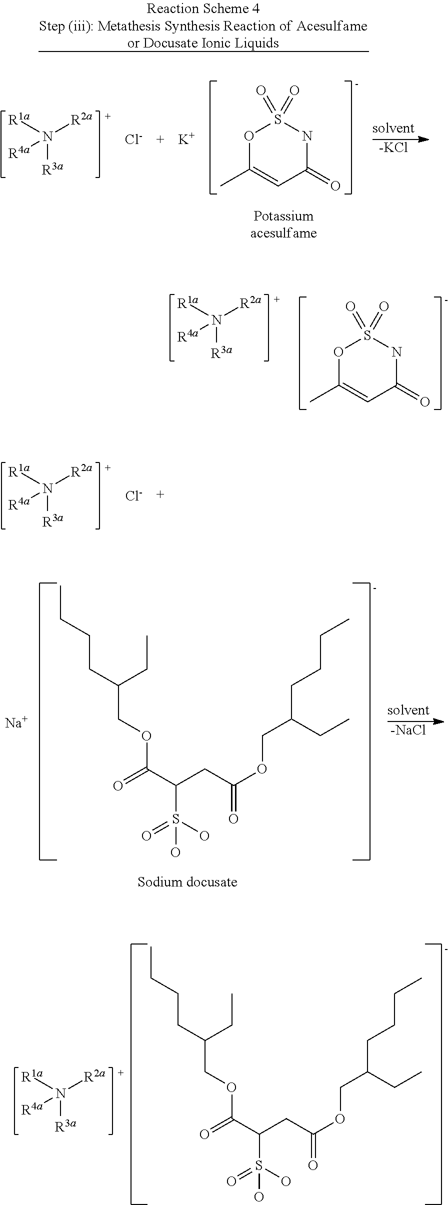

To a chloride ionic liquid in dichloromethane, potassium acesulfame or sodium docusate are added in equimolar quantities, followed by sonication and stirring for 6 h. The byproduct, potassium or sodium chloride, is removed by centrifugation at 4,400 rpm, followed by filtration. The solvent is removed via rotary evaporation. The resulting product is dried by heating at 40-80° C. for 1-3 days, under high vacuum.

TABLE 3
Exemplary Ionic Liquids with Acesulfame or Docusate Anion from Metathesis Reaction

| Example | Chemical Name | Chemical Structure |
|---|---|---|
| Ionic Liquid 7 | (N-ethyl-2- (2-methoxyethoxy)- N,N-dimethyl- ethanaminium) 6- methyl-3,4- dihydro-1,2,3- oxathiazin-4- | 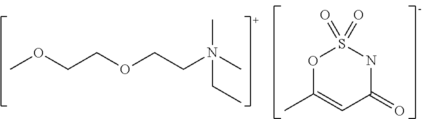 |
| | one 2,2-dioxide | |
| Ionic Liquid 8 | 2-(2-ethoxyethoxy)- N-ethyl-N,N- dimethyl- ethanaminium 6- methyl-3,4- dihydro-1,2,3- oxathiazin-4- | 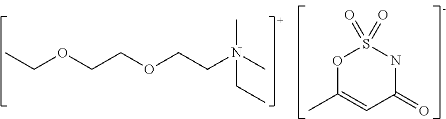 |
| | one 2,2-dioxide | |
| Ionic Liquid 9 | N-benzyl-N,N- dimethylnonan-1- aminium 6-methyl- 3,4-dihydro- 1,2,3-oxathiazin- 4-one 2,2- dioxide | 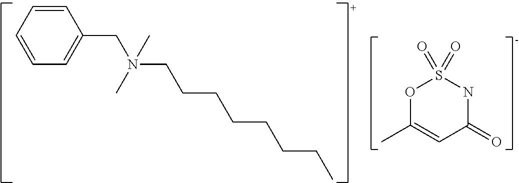 |
| Ionic Liquid 10 | (N-ethyl-2- (2-methoxyethoxy)- N,N-dimethyl- ethanaminium) 1,4- bis(2-ethylhexoxy)-1,4- dioxobutane- 2-sulfonate | 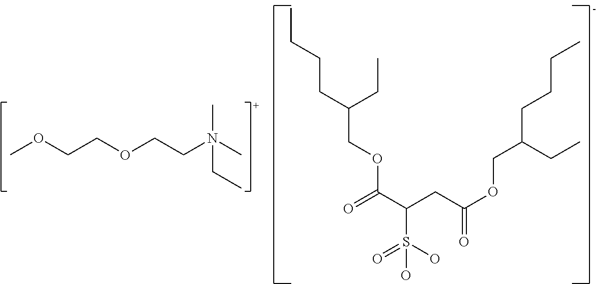 |
| Ionic Liquid 11 | 2-(2-ethoxyethoxy)- N-ethyl-N,N- dimethylethanaminium 1,4- bis(2- ethylhexoxy)- 1,4- dioxobutane- 2-sulfonate | 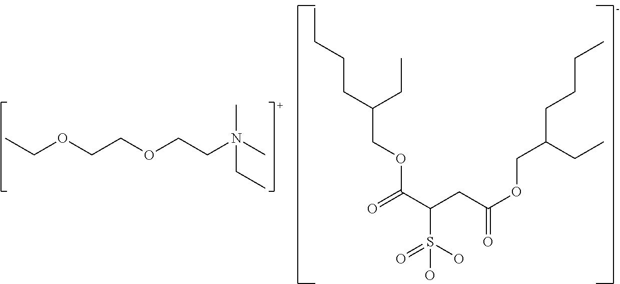 |
| Ionic Liquid 12 | N-benzyl-N,N- dimethylnonan-1- aminium 1,4-bis(2-ethylhexoxy)- 1,4-dioxobutane- 2-sulfonate | 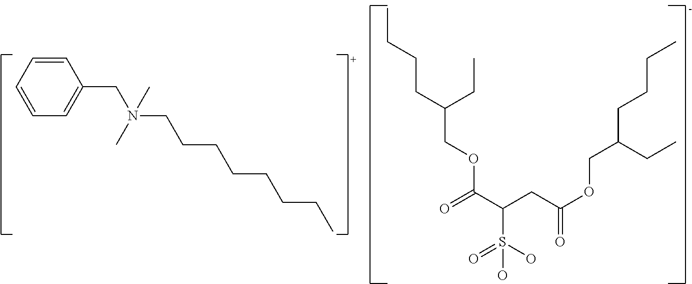 |

### Example 2

### Characterisation Data for Ionic Liquids

The characterisation data for the exemplary ionic liquids are provided in Tables 4, 5 and 6.

TABLE 4
ES-MS and Elemental Analysis Data

| | Elem. | | | | | | | | |
|---|---|---|---|---|---|---|---|---|---|
| Ionic | Anal. | | | | | | | ES-MS | ES-MS |
| Liquid | (%) | C | H | N | S | K | Cl | (M⁺) | (M⁻) |
| Ionic | Calcd | 51.05 | 10.47 | 6.62 | — | — | — | 176.1651 | — |
| Liquid 1 | Measd. | 48.24 | 10.6 | 5.95 | — | — | — | 176.1624 | — |
| Ionic | Calcd. | 53.20 | 10.72 | 6.20 | — | — | — | 190.1807 | — |
| Liquid 2 | Measd. | 52.40 | 10.82 | 6.25 | — | — | — | 190.1782 | — |
| Ionic | Calcd. | 52.75 | 10.33 | 4.10 | — | — | — | 246.2433 | 94.9803 |
| Liquid 3 | Measd. | 51.86 | 10.36 | 4.24 | — | — | — | 246.2417 | 94.9803 |
| Ionic | Calcd. | 71.93 | 10.65 | 4.93 | — | — | — | 248.2378 | — |
| Liquid 4 | Measd. | 70.70 | 10.99 | 4.77 | — | — | — | 248.2375 | — |
| Ionic | Calcd. | 46.29 | 9.54 | 4.91 | 11.24 | | 0 | 190.1807 | 94.9803 |
| Liquid 5 | Measd. | 44.61 | 9.47 | 7.75 | 11.04 | | 1.46 | 190.1784 | 94.9796 |
| Ionic | Calcd. | 58.26 | 9.95 | 2.34 | 5.36 | 0 | 0 | 176.1651 | 161.9861 |
| Liquid 7 | Measd. | 57.01 | 10.3 | 2.27 | 4.70 | 0.01 | 0.13 | 176.1555 | 161.9668 |
| Ionic | Calcd. | 47.71 | 8.01 | 7.95 | 9.10 | 0 | 0 | 190.1807 | 161.9861 |
| Liquid 8 | Measd. | 47.34 | 8.08 | 7.98 | 8.43 | 0 | 0.45 | 190.1755 | 161.9861 |
| Ionic | Calcd. | 61.43 | 8.35 | 6.82 | 7.81 | 0 | 0 | 248.2378 | 161.9861 |
| Liquid 9 | Measd. | 61.47 | 8.67 | 6.91 | 7.67 | 0 | 0.41 | 248.2382 | 161.9829 |
| Ionic | Calcd. | 58.26 | 9.95 | 2.34 | 5.36 | 0 | 0 | 176.1651 | 421.2260 |
| Liquid | Measd. | 57.61 | 9.88 | 2.41 | — | 0.07 | 0.5 | 176.1628 | 421.2107 |
| 10 | | | | | | | | | |
| Ionic | Calcd. | 47.71 | 8.01 | 7.95 | 9.10 | 0 | 0 | 190.1807 | 421.2260 |
| Liquid | Measd. | 47.34 | 8.08 | 7.98 | 8.45 | 0.03 | 1.1 | 190.1836 | 421.2171 |
| 11 | | | | | | | | | |
| Ionic | Calcd. | 66.35 | 10.08 | 2.09 | 4.79 | 0 | 0 | 248.2378 | 422.2260 |
| Liquid | Measd. | 66.55 | 10.15 | 2.19 | 4.94 | 0.2 | 0.5 | 248.2366 | 422.2258 |
| 12 | | | | | | | | | |

TABLE 5
NMR Data

| Ionic Liquid | NMR Data |
|---|---|
| Ionic Liquid 1 | ¹H NMR (δ_{H}/ppm, 400 MHz, CDCl₃): 3.98 (m, 2H); 3.94 (m, 2H); |
| | 3.80-3.79 (quartet, 2H, J = 4 Hz); 3.67 (m, 2H); 3.52 (m, 2H); 3.42 (s, 6H); |
| | 3.36 (s, 3H); 1.42 (t, 3H). |
| Ionic Liquid 2 | ¹H NMR (δ_{H}/ppm, 400 MHz, D₂O): 3.88 (m, 2H); 3.62 (m, 4H); |
| | 3.54-3.48 (m, 4H); 3.41-3.39 (quart, 2H, J = 8 Hz); 3.04 (s, 6H); 1.29 (t, 3H); |
| | 1.12 (t, 3H). |
| Ionic Liquid 3 | ¹H NMR (δ_{H}/ppm, 400 MHz, CDCl₃): 3.96 (s, 2H), 3.79 (m, 2H), |
| | 3.69-3.62 (m, 4H), 3.53-3.49 (m, 2H), 3.34 (s, 3H), 3.28 (s, 6H), 2.73 (s, |
| | 3H), 2.00 (s, 2H), 1.40 (t, 3H). Tg = −83° C. |
| Ionic Liquid 4 | ¹H NMR (δ_{H}/ppm, 400 MHz, CDCl₃) δ 7.70-7.64 (m, 2H), |
| | 7.50-7.40 (m, 3H), 5.09 (s, 2H), 3.56-3.48 (m, 2H), 3.32 (s, 6H), 2.02 (s, |
| | 1H), 1.80 (s, 2H), 1.29 (m, J = 20.5, 13.0 Hz, 10H), 0.88 (t, J = 6.8 Hz, |
| | 3H). m.p = 62° C. |
| Ionic Liquid 5 | ¹H NMR (δ_{H}/ppm, 400 MHz, CDCl₃) δ 3.96 (s, 2H), 3.81-3.74 (m, |
| | 2H), 3.67 (m, 4H), 3.57-3.44 (m, 4H), 3.29 (s, 6H), 2.72 (s, 3H), |
| | 1.41 (t, J = 7.2 Hz, 3H), 1.18 (t, J = 7.0 Hz, 3H). |
| Ionic Liquid 7 | ¹H NMR (δ_{H}/ppm, 400 MHz, CDCl₃): 5.42 (s, 1H); 3.94 (m, 2H); |
| | 3.71 (m, 2H); 3.64-3.62 (m, 4H); 3.51 (m, 2H); 3.34 (s, 3H); 3.23 (s, 6H); |
| | 2.00 (s, 3H); 1.39 (t, 3H). Tg = −66.6° C. |
| Ionic Liquid 8 | ¹H NMR (δ_{H}/ppm, 400 MHz, D₂O) δ 5.60 (s, 1H), 3.88 (s, 2H), 3.62 (s, |
| | 4H), 3.58-3.44 (m, 4H), 3.39 (q, J = 7.3 Hz, 2H), 3.04 (s, 6H), 2.05 (s, |
| | 3H), 1.29 (t, 3H), 1.12 (t, 3H). |
| Ionic Liquid 9 | ¹H NMR (δ_{H}/ppm, 400 MHz, CDCl₃) δ 7.70-7.64 (m, 2H), |
| | 7.50-7.40 (m, 3H), 5.09 (s, 2H), 3.56-3.48 (m, 2H), 3.32 (s, 6H), 2.02 (s, 1H), |
| | 1.80 (s, 2H), 1.29 (m, 10H), 0.88 (t, J = 6.8 Hz, 3H). m.p. = 55° C. |
| Ionic Liquid 10 | ¹H NMR (δ_{H}/ppm, 400 MHz, CDCl₃) δ 4.12 (m, 1H), 4.10-3.91 (m, |
| | 6H), 3.75 (s, 2H), 3.65 (m, 4H), 3.54 (s, 2H), 3.38 (s, 3H), 3.25 (s, 7H), |
| | 3.12 (m, 1H), 1.74 (s, 2H), 1.65 (s, 1H), 1.54 (s, 1H), 1.50-1.22 (m, |
| | 20H), 0.89 (m, 12H). Tg = −81° C. |
| Ionic Liquid 11 | ¹H NMR (δ_{H}/ppm, 400 MHz, CDCl₃): ¹H NMR (400 MHz, CDCl₃) δ |
| | 4.15-3.86 (m, 6H), 3.81-3.71 (m, 2H), 3.71-3.59 (m, 4H), |
| | 3.59-3.43 (m, 4H), 3.31-3.16 (m, 7H), 3.09 (m, 1H), 2.07 (s, 1H), 1.57 (m, |
| | 2H), 1.46-1.19 (m, 17H), 1.18 (m, 2H), 0.96-0.80 (m, 10H). |
| Ionic Liquid 12 | ¹H NMR (δ_{H}/ppm, 400 MHz, CDCl₃) δ 7.58 (s, 2H), 7.44 (s, 3H), |
| | 4.72 (s, 2H), 4.16 (m, 1H), 3.97 (m, 4H), 3.31 (m, 3H), 3.15 (m, 7H), 1.60 (s, |
| | 1H), 1.51 (s, 1H), 1.45-1.17 (m, 26H), 0.93-0.79 (m, 15H). |

TABLE 6
Viscosity Data

| Ionic Liquid | Water/%¹ | Viscosity/Pa · s |
|---|---|---|
| Ionic Liquid 3 | 0.348 | 0.9197 |
| Ionic Liquid 5 | 0.607 | 0.9029 |
| Ionic Liquid 7 | 0.550 | 0.7346 |
| Ionic Liquid 8 | 0.111 | 0.8266 |
| Ionic Liquid 10 | 0.092 | 4.7860 |
| Ionic Liquid 11 | 0.353 | 2.4730 |
| Ionic Liquid 12 | —² | 33.690 |

¹Karl Fischer measurements carried out on a Cou-Lo Compact (GRScientific) apparatus. This automated method directly measures the water content of a material using a coulometric titration.
²Sample too viscous for Karl Fisher measurement.

### Example 3

### Compositions

The following are non-limiting examples of compositions containing ionic liquids of the present invention. They are prepared by admixture of the components described in the tables, in the proportions indicated.

TABLE 7
A Liquid Fabric Softener Composition

| Ingredients | Amounts (wt %) |
|---|---|
| Di(hydrogenated tallow)dimethyl ammonium chloride | 5.25 |
| Perfume | 2.00 |
| Ionic liquid¹ | 0.5 |
| Water | To 100 |

¹Any exemplary Ionic Liquid 1 to Ionic Liquid 12 or mixtures thereof.

TABLE 8
A Liquid Laundry Detergent Composition

| Ingredients | Amounts (wt %) |
|---|---|
| K/Na C13 linear alkylbenzen sulfonate | 7.2 |
| K/Na C14-15 alkyl polyethoxylate(2.25)sulfonate | 10.8 |
| C12-13 alcohol poly(6.5)ethoxylate | 6.5 |
| C12 alkyltrimethyl ammonium chloride | 1.2 |
| C12-14 fatty acid | 13.0 |
| Oleic acid | 2.0 |
| Citric acid (anhydrous) | 4.0 |
| Diethylenetriamine pentaacetic acid | 0.23 |
| Enzyme | 0.91 |
| Ethoxylated tetraethylene pentamine (15-18 mol. EO at | 1.5 |
| each H) | |
| Monoethanolamine | 2.0 |
| Propylene glycol | 7.25 |
| Ethanol | 7.75 |
| Formic acid | 0.66 |
| Calcium ion | 0.03 |
| Perfume | 1.0 |
| Ionic liquid¹ | 0.1 |
| Water and minors | To 100 |

¹Any exemplary Ionic Liquid 1 to Ionic Liquid 12 or mixtures thereof.

TABLE 9
Air Care Compositions

| | Amounts (wt %) | | | | | |
|---|---|---|---|---|---|---|
| Ingredients | I | II | III | IV | V | VI |
| Hydroxypropyl | 0.2 | — | — | — | 0.3 | 0.1 |
| beta-cyclo- | | | | | | |
| dextrin | | | | | | |
| Zwitterionic | 0.1 | 0.1 | 0.1 | — | 0.1 | 0.05 |
| Polymer | | | | | | |
| Diethylene | 0.25 | — | — | — | — | — |
| glycol | | | | | | |
| Silwet L-7600 | 0.1 | 0.2 | — | 0.2 | 0.1 | 0.1 |
| Sodium Dioctyl | 0.2 | — | 0.2 | 0.1 | 0.2 | 0.2 |
| Sulfosuccinate | | | | | | |
| Acid Salt | 0.1 | 0.1 | — | 0.2 | 0.1 | — |
| Ethanol | 3 | 5 | 5 | 3 | 5 | 5 |
| Hydrogenated | 0.4 | 0.8 | 1.2 | 1.6 | 1.8 | 5 |
| castor oil | | | | | | |
| Perfume | 0.6 | 0.8 | 0.4 | 0.2 | 1 | 0.1 |
| Mixture | | | | | | |
| Ionic Liquid¹ | 0.01 | 0.1 | 0.5 | 0.8 | 0.025 | 0.2 |
| Organic Acid | 0.05 | 0.1 | — | 0.1 | 0.05 | — |
| Preservative | 0.015 | 0.015 | 0.015 | 0.015 | 0.015 | 0.015 |
| HCl or NaOH | to pH 5 | to pH 5 | to pH 5 | to pH 5 | to pH 5 | to pH 5 |
| Distilled water | Balance | Balance | Balance | Balance | Balance | Balance |

¹Any exemplary Ionic Liquid 1 to Ionic Liquid 12 or mixtures thereof.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An ionic liquid composition comprising:
(a) an anion; and
(b) a cation selected from the group consisting of: 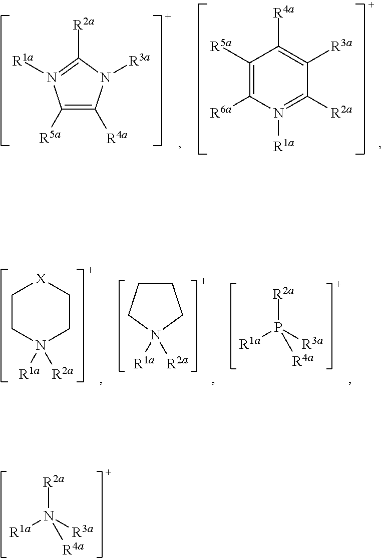
and combinations thereof;
wherein: X is CH₂ or O;
each R^{1a}, R^{3a}, and R^{4a} are independently selected from hydrogen, C₁-C₂₀ alkyl, C₂-C₂₀ alkenyl, C₂-C₂₀ alkynyl, C₁-C₂₀alkoxy, C₁-C₂₀alkoxyC₁-C₂₀alkyl, C₃-C₇cycloalkyl, C₃-C₇cycloalkylC₁-C₄alkyl, C₂-C₂₀heterocyclyl, C₆-C₁₀aryl, C₆-C₁₀arylC₁-C₁₀alkyl, C₁-C₁₀heteroaryl, halo, haloC₁-C₂₀alkyl, hydroxyl, hydroxyC₁-C₂₀alkyl, or —N(R^{6a})₂;
each R^{2a} is independently selected from hydrogen, C₁-C₂₀ alkyl, C₁-C₂₀ alkenyl, or C₁-C₂₀ alkynyl;
each R^{5a} is independently selected from hydrogen, C₁-C₂₀ alkyl, C₁-C₂₀ alkenyl, C₁-C₂₀ alkynyl, —R^{7a}—OR^{8a}, or —R^{7a}—OR^{7a}—OR^{8a};
each R^{6a} is independently selected from hydrogen, alkyl, alkenyl, alkynyl, haloalkyl, alkoxyalkyl, cycloalkyl, cycloalkylalkyl, aryl, aralkyl, heterocyclyl, heterocyclyalkyl, heteroaryl, or heteroarylalkyl;
each R^{7a} is independently selected from a direct bond, alkylene chain, alkenylene chain, or alkynylene chain; and
each R^{8a} is independently selected from a hydrogen, alkyl, alkenyl or alkynyl.

2. The composition of claim 1, wherein:
R^{1a} and R^{3a} are independently selected from C₁-C₆ alkyl;
R^{2a} is C₁-C₆ alkyl;
R^{4a} is C₁-C₂₀alkoxyC₁-C₂₀alkyl substituted with —OR¹⁰; and
R¹⁰ is C₁-C₆ alkyl.

3. The composition of claim 2, wherein the cation is (N-ethyl-2-(2-methoxyethoxy)-N,N-dimethylethanaminium); 2-(2-ethoxyethoxy)-N-ethyl-N,N-dimethylethanaminium; or mixtures thereof.

4. The composition of claim 1, wherein:
R^{1a}, R^{2a} and R^{4a} are hydrogen; and
R^{3a} and R^{5a} are C₁-C₆ alkyl.

5. The composition of claim 4, wherein that cation is N-benzyl-N,N-dimethyloctan-1-aminium; N-benzyl-N,N-dimethylnonan-1-aminium; or mixtures thereof.

6. The composition of claim 1, wherein the anion has the formula (I):
[R¹—O—C(O)—CH(SO₃)—R³—C(O)—O—R²]⁻ (I)
wherein:
R¹ and R² are independently selected from the group consisting of alkyl, mono or oligo-alkoxyalkyl and alkenyl; and
R³ is alkylene, heteroarylene, arylene, or cycloalkylene.

7. The composition of claim 6, wherein:
R¹ and R² are independently selected from alkyl having 5 or more carbon atoms, preferably 6 to 18 carbon atoms; and
R³ is —(CH₂)ₙ— wherein n is an integer of from 1 to 12.

8. The composition of claim 7, wherein the anion is 1,4-bis(2-ethylhexoxy)-1,4-dioxobutane-2-sulfonate.

9. The composition of claim 1, wherein the anion has the general formula (II): 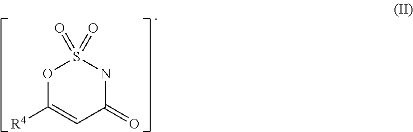
wherein:
R⁴ is selected from the group consisting of hydrogen, cyano, alkyl, alkoxy and alkoxyalkyl.

10. The composition of claim 9, wherein the anion is 6-methyl-3,4-dihydro-1,2,3-oxathiazin-4-one 2,2-dioxide.

11. The composition of claim 1, wherein the anion has the general formula (III):
[R⁹—SO₂—N—SO₂—R⁹]⁻ (III)
wherein:
R⁹ is selected from the group consisting of haloalkyl, alkyl and alkenyl.

12. The composition of claim 11, wherein the anion is bis{(trifluoromethyl)sulfonyl}amide.

13. The composition of claim 1, wherein the anion is independently selected from the group consisting of: 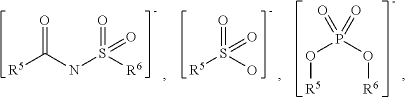
and combinations thereof;
wherein:
each R⁵ and R⁶ are independently selected from the group consisting of hydrogen, alkyl, alkenyl, alkynyl, alkoxyalkyl, cycloalkyl, cycloalkylalkyl, aryl, aralkyl, heterocyclyl, heterocyclyalkyl, heteroaryl and heteroarylalkyl.

14. The composition of claim 1, wherein the composition is a liquid at temperatures less than about 100° C., preferably at ambient temperature.

15. The composition of claim 1, wherein the cation and anion are essentially free of the following chemical elements: antimony, barium, beryllium, bromine, cobalt, chromium, fluorine, iodine, lead, nickel, selenium, thallium and mixtures thereof.

16. A method for treating a target surface comprising the step of contacting the target surface with an ionic liquid composition of claim 1.
